(19) United States
(12) Patent Application Publication
(10) Pub. No.: US 2012/0277748 A1
(43) Pub. Date: Nov. 1, 2012

(54) BONE PLATE

(75) Inventors: Paul Trescony, Champlin, MN (US); Jeffrey Rouleau, Maple Grove, MN (US); Matthew Jolly, Minneapolis, MN (US)

(73) Assignee: Warsaw Orthopedic, Inc., Warsaw, IN (US)

(21) Appl. No.: 13/096,451

(22) Filed: Apr. 28, 2011

(51) Int. Cl. A61B 17/80

(52) U.S. Cl. 606/70

(57) ABSTRACT

A plate for connecting a first portion of bone and a second portion of bone is disclosed. The plate comprises a central longitudinal axis, a first end portion extending along the central longitudinal axis and configured to affix to the first portion of bone, a second end portion extending along the central longitudinal axis and configured to affix to the second portion of bone, and an intermediary portion extending along the central longitudinal axis and between the first end portion and the second end portion. The intermediary portion comprises three continuous segments of material, two segments of which extend along a direction substantially parallel to the central longitudinal axis, and one segment of which extends along a direction substantially perpendicular to the central longitudinal axis.

### FIELD OF INVENTION

The present invention is directed to systems for connecting two portions of bone.

### BACKGROUND

The present disclosure relates to systems for connecting two portions of bone, and more particularly, flexible systems for connecting two portions of bone.

### SUMMARY OF THE INVENTION

A plate for connecting a first portion of bone and a second portion of bone is disclosed. The plate comprises a central longitudinal axis, a first end portion extending along the central longitudinal axis and configured to affix to the first portion of bone, a second end portion extending along the central longitudinal axis and configured to affix to the second portion of bone, and an intermediary portion extending along the central longitudinal axis and between the first end portion and the second end portion. The intermediary portion comprises three continuous segments of material, two segments of which extend along a direction substantially parallel to the central longitudinal axis, and one segment of which extends along a direction substantially perpendicular to the central longitudinal axis.

Further, a system for connecting a first portion of bone and a second portion of bone also is disclosed.

### BRIEF DESCRIPTION OF THE DRAWINGS

FIG. 1 is a front view of a plate for connecting a first portion of bone and a second portion of bone;

FIG. 2 is an isometric view of another plate;

FIG. 2A is a front view of the plate of FIG. 2;

FIG. 2B is a side view of the plate of FIG. 2;

FIG. 2C is a partial, enlarged side view of the plate of FIG. 2;

FIG. 2D is a partial, enlarged side view of another plate;

FIG. 2E is a partial, enlarged side view of another plate;

FIG. 2F is a partial, enlarged side view of another plate;

FIG. 2G is a partial, enlarged side view of another plate;

FIG. 2H is a partial, enlarged side view of another plate;

FIG. 2I is a partial, enlarged side view of another plate;

FIG. 3 is an isometric view of another plate;

FIG. 3A is a front view of the plate of FIG. 3;

FIG. 3B is a side view of the plate of FIG. 3;

FIG. 3C is a partial, enlarged front view of the plate of FIG. 3;

FIG. 3D is a partial, enlarged front view of another plate;

FIG. 4 is an isometric view of another plate;

FIG. 4A is a front view of the plate of FIG. 4;

FIG. 4B is a side view of the plate of FIG. 4;

FIG. 4C is a partial, enlarged front view of the plate of FIG. 4; and

FIG. 4D is a partial, enlarged front view of another plate.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

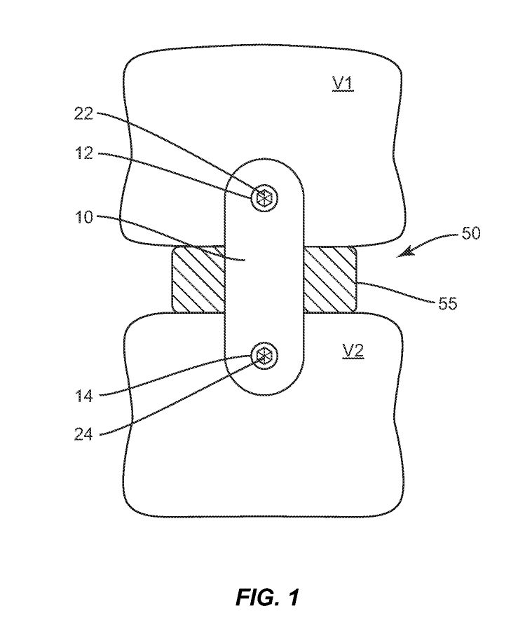

FIG. 1 shows a front view of a stratum or plate 10 for connecting a first portion of bone V1 and a second portion of bone V2. As shown in FIG. 1, the plate 10 represents a spinal plate for affixing two vertebral bodies, for example, the first portion of bone V1 represents a first vertebral body V1 and the second portion of bone V2 represents a second vertebral body V2. As shown in FIG. 1, the plate 10 may be used to affix or fuse two adjacent vertebral bodies, for example, the superior vertebral body V1 and the inferior vertebral body V2. As shown in FIG. 1, the plate 10 comprises a central longitudinal axis CLA, which parallels the long axis of the plate 10. Typically, as shown, the long axis of the plate 10 is substantially parallel to the longitudinal axis of the spine.

The term “substantially” as used herein may be applied to modify any quantitative representation which could permissibly vary without resulting in a change in the basic function to which it is related. For example, the long axis of a plate may be considered substantially parallel to the longitudinal axis of the spine even though the longitudinal axis of the plate may be slightly different than that of the spine. For example, in the cervical spine, a plate may be pre-lordosed to attempt to match the anatomical curvature of the location on the spine the plate is to be placed, but exact matches over the entire length of the plate are difficult to achieve. Similarly, the longitudinal axis of the spine varies over the length of a spine.

As shown in FIG. 1, natural disc tissue between the two adjacent vertebral bodies V1 and V2 has been removed, leaving a disc space 50. An interbody device 55 such as a spacer may be implanted between vertebral bodies V1 and V2 and a spinal plate such as plate 10 may be used, for example, to fuse the vertebral bodies V1 and V2 and also may accomplish the function of helping to maintain the interbody device 50 in place between vertebral bodies V1 and V2.

As shown in FIG. 1, the plate 10 further comprises a first fastener hole 12 for receiving a first fastener 22 and a second fastener hole 14 for receiving a second fastener 24. Each fastener has a head and a shaft (not shown). In cases where the plate 10 is a bone plate or a spinal plate, as shown in FIG. 1, the fasteners may be bone screws.

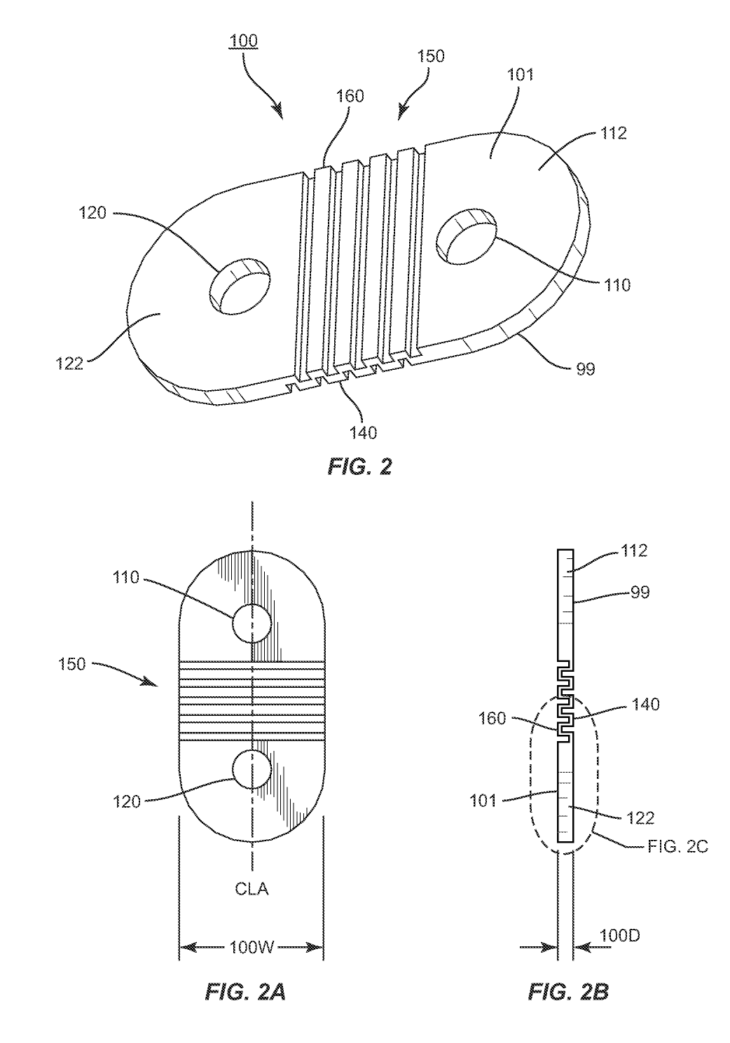

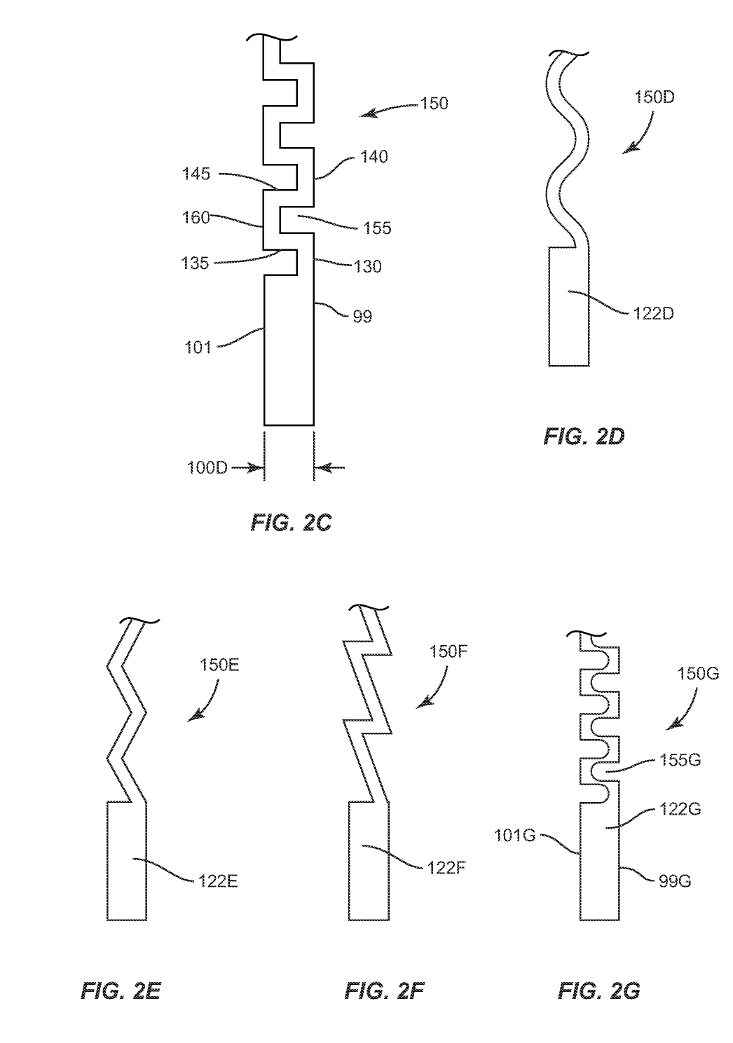

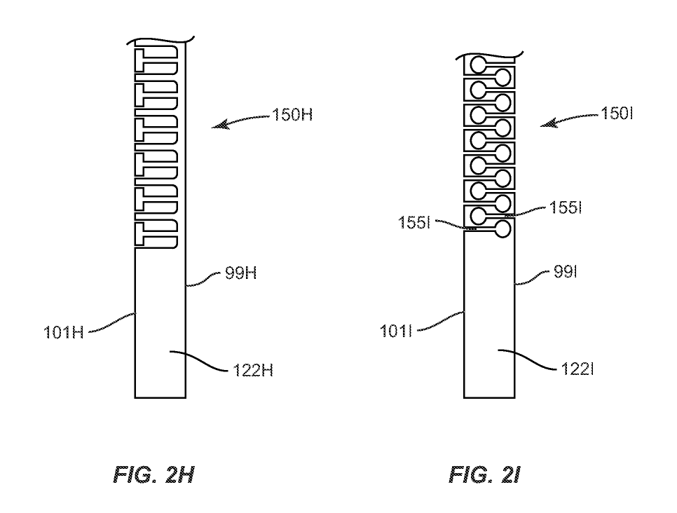

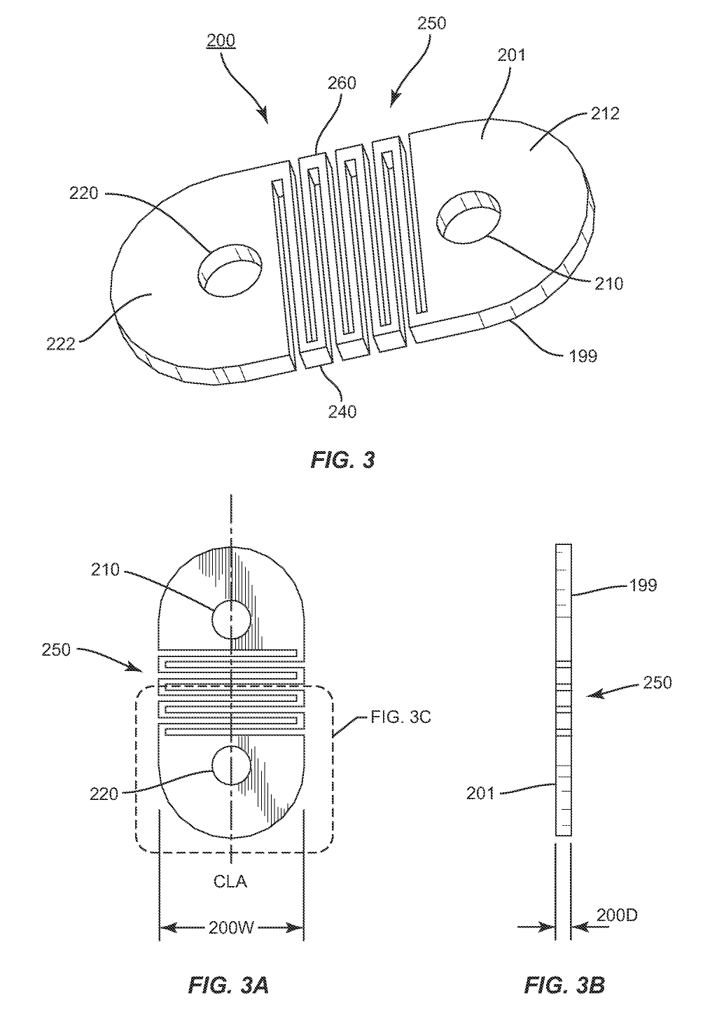

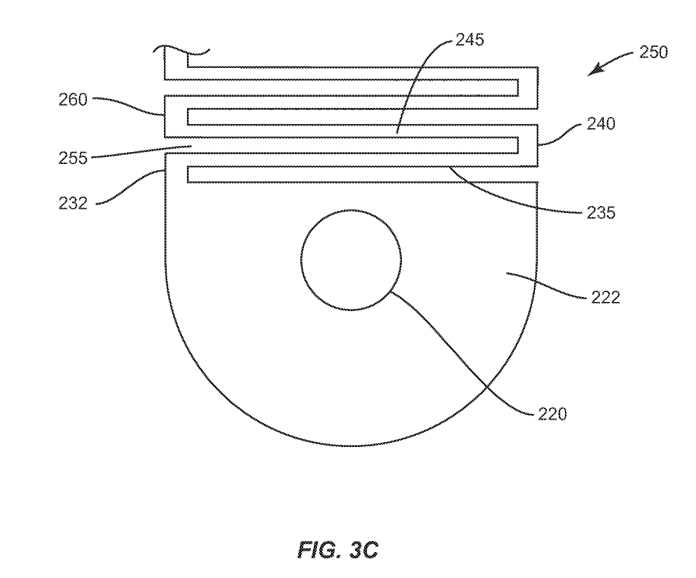

FIG. 2 shows an isometric view of a plate 100. As shown in FIG. 2, plate 100 is designed to connect a first portion of bone and a second portion of bone. As shown in FIG. 2, the plate 100 comprises a central longitudinal axis CLA, a first end portion 112 extending along the central longitudinal axis CLA and configured to affix to the first portion of bone, and a second end portion 122 extending along the central longitudinal axis CLA and configured to affix to the second portion of bone. As shown in FIG. 2, the plate 100 further comprises an intermediary portion 150 extending along the central longitudinal axis CLA and between the first end portion 112 and the second end portion 122, wherein the intermediary portion 150 comprises a plurality of continuous segments (such as segment 140) of material.

FIG. 2A shows a front view of the plate 100 of FIG. 2, and FIG. 2B shows a side view of the plate 100 of FIG. 2. As shown in FIGS. 2 and 2A, the first end portion 112 has a fastener hole 110 for receiving a first bone-engaging fastener, and the second end portion 122 has a fastener hole 120 for receiving a second bone-engaging fastener. Note that although each of the first and second end portions 112 and 122 have only one fastener hole, one or both of each end portion of plate 100 may comprise more than one fastener hole. As shown in FIGS. 2 and 2B, plate 100 further comprises a first surface 99 for engaging bone, a second surface 101 opposing the first surface, and a depth 100D defined by a distance between the first surface 99 and the second surface 101.

FIG. 2C shows a partial, enlarged side view of the plate 100, and more specifically, the area 2C of the plate 100 as shown in FIG. 2B. As shown in FIG. 2C, the intermediary portion 150 of plate 100 comprises a plurality of continuous segments (such as segment 140) of material. As shown in FIG. 2C, two segments, for example segments 140 and 160 extend along a direction substantially parallel to the central longitudinal axis CLA, and one segment, for example, segment 145 extends along a direction substantially perpendicular to the central longitudinal axis CLA.

As shown in FIGS. 2 and 2A, the first end portion 112, the second end portion 122 and the intermediary portion 150 have a width 100W extending in a direction substantially perpendicular to the central longitudinal axis CLA of the plate 100. As shown in the figures, and particularly FIG. 2C, at least one segment, for example, segment 145 of the intermediary portion 150 extends along a direction substantially perpendicular to the central longitudinal axis CLA and extends along a direction substantially perpendicular to the width 100W of the intermediary portion 150. Further, as shown in the figures, and particularly FIG. 2C, at least one segment, for example, segment 145 of the intermediary portion 150 extends along a direction substantially perpendicular to the central longitudinal axis extends CLA in a direction substantially parallel to the depth 100D of the plate 100.

As shown in FIG. 2C, the intermediary portion 150 comprises a plurality of continuous segments, for example, a first segment such as segment 130 that extends along a direction substantially parallel to the central longitudinal axis CLA, and a second segment such as segment 135 that extends along a direction substantially perpendicular to the central longitudinal axis CLA and perpendicular to the width 100W of the intermediary portion 150, wherein the second segment 135 is connected to the first segment 130. Further, the intermediary portion 150 further comprises a third segment such as segment 160 that extends along a direction substantially parallel to the central longitudinal axis CLA, and a fourth segment such as segment 145 that extends along a direction substantially perpendicular to the central longitudinal axis CLA and perpendicular to the width 100W of the intermediary portion 150, wherein the third segment 160 is connected to the second segment 135 and wherein the fourth segment 145 is connected to the third segment 160.

Similarly, as shown in FIG. 2C, the intermediary portion 150 comprises a plurality of continuous segments, for example, a first segment such as segment 130 that extends along a direction substantially parallel to the central longitudinal axis CLA, and a second segment such as segment 135 that extends along a direction substantially perpendicular to the central longitudinal axis CLA and substantially parallel to the depth 100D of the plate 100, wherein the second segment 135 is connected to the first segment 130. Further, intermediary portion 150 further comprises a third segment such as segment 160 that extends along a direction substantially parallel to the central longitudinal axis CLA, and a fourth segment such as segment 145 that extends along a direction substantially perpendicular to the central longitudinal axis CLA and substantially parallel to the depth 100D of the plate, wherein the third segment 160 is connected to the second segment 135 and wherein the fourth 145 segment is connected to the third segment 160.

Also, as shown in FIG. 2B and FIG. 2C, the second segment 135 extends away from the first segment 130 in a direction away from the first surface 99 of the plate 100, and the fourth segment 145 extends away from the third segment 160 in a direction toward the first surface 99 of the plate. As shown, the first segment 130 makes up part of the first surface 99 of the plate 100, whereas the third segment 160 makes up part of the second surface 101 of the plate 100.

As shown in FIG. 2, for example, the intermediary portion 150 of plate 100 comprises more than four continuous segments. For example, as shown in FIG. 2, the intermediary portion 150 further comprises fifth, sixth, seventh and eighth segments that are situated in the same manner as the first, second, third and fourth segments, respectively, wherein the fifth segment is connected to the fourth segment. More specifically, the second segment such as segment 135 extends away from the first segment such as segment 130 in a direction away from the first surface 99 of the plate 100, the fourth segment such as segment 145 extends away from the third segment such as segment 160 in a direction toward the first surface 99 of the plate 100, the sixth segment extends away from the fifth segment in a direction away from the first surface 99 of the plate 100, and the eighth segment extends away from the seventh segment in a direction toward the first surface 99 of the plate 100.

The arrangements of the continuous segments of the intermediary portion 150 described herein help provide flexibility to the plate 100 that otherwise may not be present. As shown in FIG. 2C, the arrangement of the segments leave spaces such as space 155 between the undulations of the continuous segments, thereby rendering the intermediary portion 150 thinner than the first and second end portions 112 and 122. In other words, the average depth of the intermediary portion 150 is less than the average depth of the other portions of the plate 100. When an analogy is made to electrical signals, as shown in FIG. 2C, a cross section of the intermediary portion 150 comprises a shape substantially similar to that of a square wave.

Alternate intermediary portions are shown in FIGS. 2D, 2E and 2F. FIG. 2D shows a partial, enlarged side view of another plate. More specifically, FIG. 2D shows a partial cross section of an intermediary portion 150D and a second end portion 122D. When an analogy is made to electrical signals, as shown in FIG. 2D, a cross section of the intermediary portion 150D comprises a shape substantially similar to that of a sine wave.

FIG. 2E shows a partial, enlarged side view of another plate. More specifically, FIG. 2E shows a partial cross section of an intermediary portion 150E and a second end portion 122E. When an analogy is made to electrical signals, as shown in FIG. 2D, a cross section of the intermediary portion 150E comprises a shape substantially similar to that of a triangle wave.

FIG. 2F shows a partial, enlarged side view of another plate. More specifically, FIG. 2F shows a partial cross section of an intermediary portion 150F and a second end portion 122F. When an analogy is made to electrical signals, as shown in FIG. 2F, a cross section of the intermediary portion 150F comprises a shape substantially similar to that of a saw tooth wave.

As shown in the figures, a variety of shapes of intermediary portions are contemplated and possible. A different shape may provide different performance characteristics of the plate, for example, one shape may provide more flexibility while another shape may provide less flexibility. Further, some shapes may be easier to manufacture and some shapes have improved fatigue performance. As another example, a cross section of an intermediary portion may appear similar to that of FIG. 2C, but the corners where two continuous segments meet may be rounded instead of sharp. A variation of this is shown in FIG. 2G, which shows a partial, enlarged side view of another plate. More specifically, FIG. 2G shows a partial cross section of an intermediary portion 150G and a second end portion 122G, wherein the interior corners of the undulations of the segments (those not adjacent the sides 99G and 101G of plate 100G) are curved.

As yet another example, a cross section of an intermediary portion may be asymmetric over the depth (or thickness) of a plate. That is, a plate may be designed such that the plate may be more (or less) flexible near the surface of the bone (for example, adjacent a first surface 99) than on the surface opposing the bone (for example, adjacent a second surface 101). For example, FIG. 2H shows a partial, enlarged side view of another plate. More specifically, FIG. 2H shows a partial cross section of an intermediary portion 150H and a second end portion 122H, wherein the depth of the plate is more flexible adjacent surface 101H than surface 99H. Note that depending on the design criteria, a plate may be designed with opposite characteristics. Similarly, many different designs of asymmetric patterns for the depth of a plate are contemplated and possible.

FIG. 2I shows a partial, enlarged side view of another plate. More specifically, FIG. 2I shows a partial cross section of an intermediary portion 150I and a second end portion 122I, wherein the depth of the plate is not necessarily more flexible adjacent surface 101I than surface 99I. As shown in FIG. 2I, the intermediary portion 150I has a built-in feature that limits the extent of compression. For example, the segments that comprise intermediary portion 150I may compress only to the extent that the spaces 155I between the segments allow. Such a design may prevent a plate from reaching critical yields or limits.

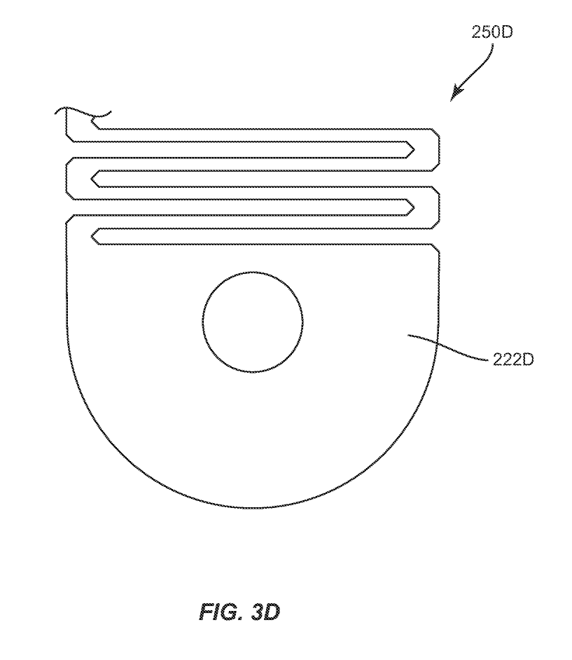

FIG. 3 shows an isometric view of a plate 200. As shown in FIG. 3, plate 200 is designed to connect a first portion of bone and a second portion of bone. Plate 200 is similar to plate 100 except that plate 200 has a different intermediary portion 250. Specifically, where the continuous segments of intermediary portion 150 undulate in a direction substantially perpendicular to the width 100D of plate 100, the continuous segments of intermediary portion 250 undulate in a direction substantially parallel to the width of plate 200.

FIG. 3A shows a front view of the plate 200 of FIG. 3, and FIG. 3B shows a side view of the plate 200 of FIG. 3. As shown, the plate 200 comprises a central longitudinal axis CLA, a first end portion 212 extending along the central longitudinal axis CLA and configured to affix to the first portion of bone, and a second end portion 222 extending along the central longitudinal axis CLA and configured to affix to the second portion of bone. As shown, the plate 200 further comprises the intermediary portion 250 extending along the central longitudinal axis CLA and between the first end portion 212 and the second end portion 222, wherein the intermediary portion 250 comprises a plurality of continuous segments (such as segment 240) of material.

As shown in FIGS. 3 and 3A, the first end portion 212 has a fastener hole 210 for receiving a first bone-engaging fastener, and the second end portion 222 has a fastener hole 220 for receiving a second bone-engaging fastener. Note that although each of the first and second end portions 212 and 222 have only one fastener hole, one or both of each end portion of plate 200 may comprise more than one fastener hole. As shown in FIGS. 3 and 3B, plate 200 further comprises a first surface 199 for engaging bone, a second surface 201 opposing the first surface, and a depth 200D defined by a distance between the first surface 199 and the second surface 201.

FIG. 3C shows a partial, enlarged front view of the plate 200, and more specifically, the area 3C of the plate 200 as shown in FIG. 3A. As shown in FIG. 3C, the intermediary portion 250 of plate 200 comprises a plurality of continuous segments (such as segment 240) of material. As shown in the figures, the continuous segments of intermediary portion 250 undulate in a direction substantially parallel to the width 200W of plate 200, and the depth 200D of the plate 200 remains substantially constant throughout the length of the intermediary portion 250. With plate 100, the continuous segments of intermediary portion 150 undulate in a direction substantially perpendicular to the width 100D of plate 100, and while the width 100W remains substantially constant over the length of intermediary portion 150, the depth 100D varies over the length of intermediary portion 150. By contrast, with plate 200, where the continuous segments of intermediary portion 250 undulate in a direction substantially parallel to the width of plate 200, the depth 200D remains substantially constant over the length of intermediary portion 250, and the width 200W varies over the length of intermediary portion 150.

That is, as shown in FIG. 3C, a first segment 232 extends from the second end portion 222 in a direction substantially parallel with the central longitudinal axis CLA and substantially perpendicular to the width 200W. A second segment 235 extends from the first segment 232 in a direction substantially perpendicular to the central longitudinal axis CLA and substantially parallel to the width 200W. A third segment 240 extends from the second segment 235 in substantially the same direction as the first segment 232. A fourth segment 245 extends from the third segment 240 in substantially the same direction as the second segment 235. A fifth segment 260 extends from the fourth segment 245 in substantially the same direction as the first segment 232. This pattern for the continuous segments of intermediary portion 250 repeats until the intermediary portion 250 reaches the first end portion 212. A set number of undulations, however, are not mandated, as the number may vary and the shape of the continuous segments may vary to achieve desired results, for example, different performance characteristics. As shown in FIG. 3C, the arrangements of continuous segments of intermediary portion 250 leave spaces such as space 255 between the undulations of the continuous segments, thereby rendering the intermediary portion 250 as having an average width less than that of the widest section of each of the first and second end portions 212 and 222. More specifically, the second segment 235, the third segment 240 and the fourth segment 245 create an undulation that yields a relatively large space 255 in the width of plate 200 at that location. As shown in the figures, space 255 spans approximately 90% of the overall width 200W of plate 200.

As stated, a variety of shapes of intermediary portions are contemplated and possible. As another example, an intermediary portion may be similar to that of FIG. 3C, but the corners where two continuous segments meet may be rounded instead of sharp. For example, FIG. 3D shows a partial, enlarged front view of another plate, which has an alternate intermediary portion 250D than that of plate 200. More specifically, instead of meeting at right angles to each other, the segments of intermediary portion 250D meet at angles that are less abrupt. That is, segments of intermediary portion 250D attempt to approximate a curved junction in that they have approximately 45 degree transition angles instead of the substantially 90 degree angles shown in intermediary portion 250 of plate 200.

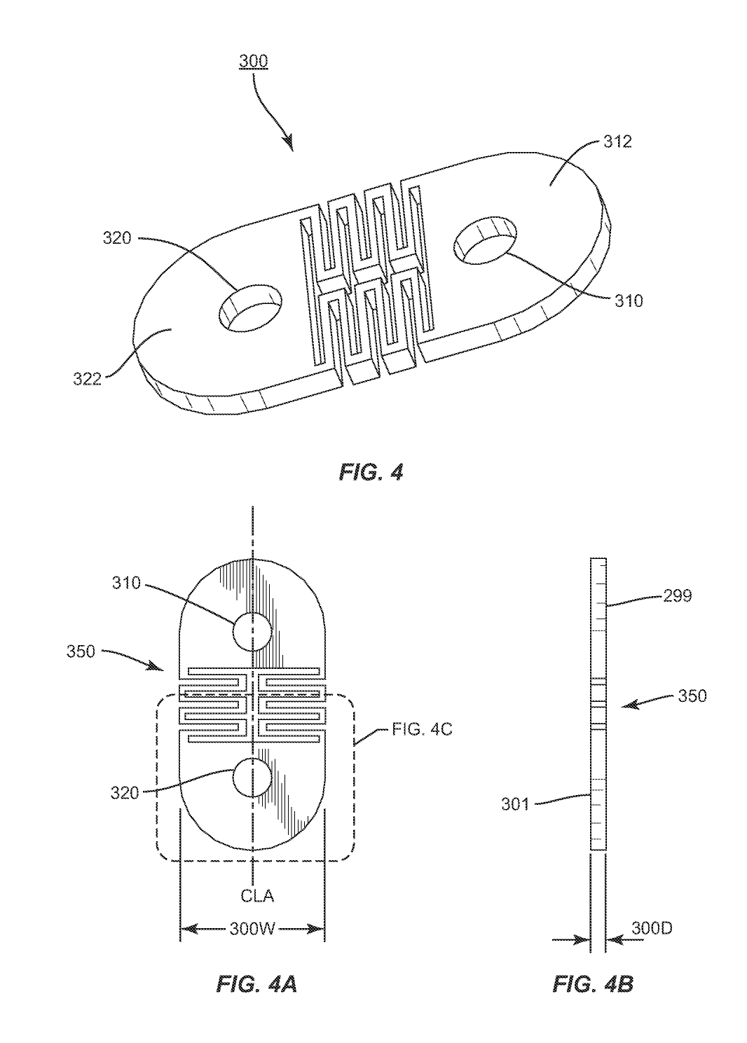

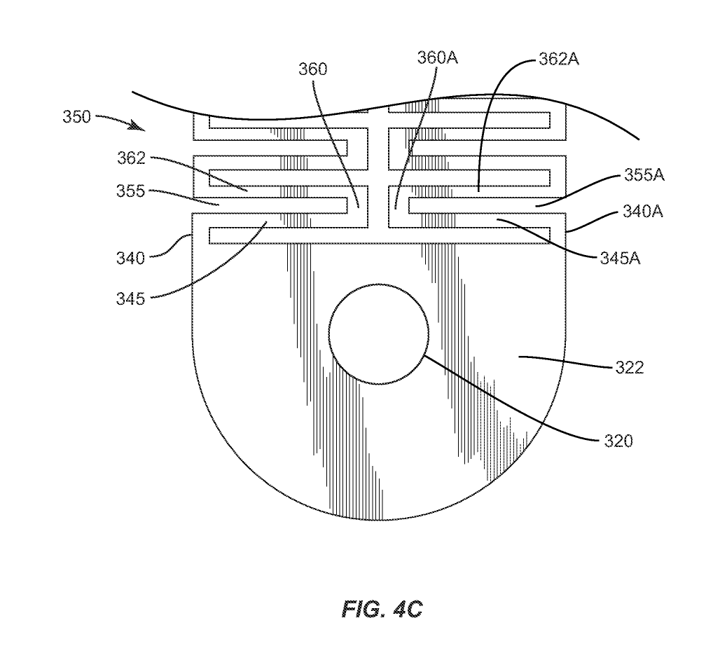

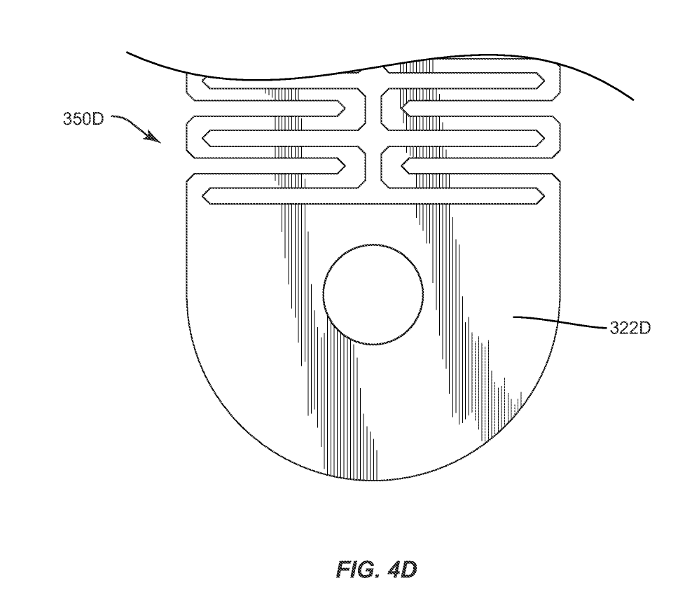

FIG. 4 shows an isometric view of a plate 300. As shown in FIG. 4, plate 300 is designed to connect a first portion of bone and a second portion of bone. Plate 300 is similar to plate 200 except that plate 300 has a different intermediary portion 350. Specifically, as with intermediary portion 250, the continuous segments of intermediary portion 350 undulate in a direction substantially parallel to the width of plate 200, but where the undulations and resulting spaces of plate 200 extended almost its entire width 200W, the undulations and resulting spaces of plate 300 extend almost half of the width of plate 300.

FIG. 4A shows a front view of the plate 300 of FIG. 4, and FIG. 4B shows a side view of the plate 300 of FIG. 4. As shown, the plate 300 comprises a central longitudinal axis CLA, a first end portion 312 extending along the central longitudinal axis CLA and configured to affix to the first portion of bone, and a second end portion 322 extending along the central longitudinal axis CLA and configured to affix to the second portion of bone. As shown, the plate 300 further comprises the intermediary portion 350 extending along the central longitudinal axis CLA and between the first end portion 312 and the second end portion 322, wherein the intermediary portion 350 comprises a plurality of continuous segments (such as segment 340) of material.

As shown in FIGS. 4 and 4A, the first end portion 312 has a fastener hole 310 for receiving a first bone-engaging fastener, and the second end portion 322 has a fastener hole 320 for receiving a second bone-engaging fastener. Note that although each of the first and second end portions 312 and 322 have only one fastener hole, one or both of each end portion of plate 300 may comprise more than one fastener hole. As shown in FIGS. 4 and 4B, plate 300 further comprises a first surface 299 for engaging bone, a second surface 301 opposing the first surface, and a depth 300D defined by a distance between the first surface 299 and the second surface 301.

FIG. 4C shows a partial, enlarged front view of the plate 300, and more specifically, the area 4C of the plate 300 as shown in FIG. 4A. As shown in FIG. 4C, the intermediary portion 350 of plate 300 comprises a plurality of continuous segments (such as segment 340) of material. As shown in the figures, the continuous segments of intermediary portion 350 undulate in a direction substantially parallel to the width 200W of plate 200, and the depth 300D of the plate 300 remains substantially constant throughout the length of the intermediary portion 350. As with plate 200, where the continuous segments of intermediary portion 350 undulate in a direction substantially parallel to the width 300W of plate 300, the depth 300D remains substantially constant over the length of intermediary portion 350, and the width 200W varies over the length of intermediary portion 350.

As shown in FIG. 4C, and particularly on the left side of FIG. 4C, a first segment 340 extends from the second end portion 322 in a direction substantially parallel with the central longitudinal axis CLA and substantially perpendicular to the width 200W. A second segment 345 extends from the first segment 340 in a direction substantially perpendicular to the central longitudinal axis CLA and substantially parallel to the width 300W. A third segment 360 extends from the second segment 345 in substantially the same direction as the first segment 340. A fourth segment 362 extends from the third segment 360 in substantially the same direction as the second segment 345. Similarly, this pattern of continuous segments is matched in “mirror image” form to that just described. That is, the right side of FIG. 4C shows segments 340A, 345A, 360A and 362A arranged in a pattern similar to, but “mirror image” of segments 340, 345, 360 and 362. This pattern for the continuous segments of intermediary portion 350 repeats until the intermediary portion 350 reaches the first end portion 312. As with the other plates described herein, a set number of undulations, however, are not mandated, as the number may vary and the shape of the continuous segments may vary to achieve desired results, for example, different performance characteristics. As shown in FIG. 4C, the arrangements of continuous segments of intermediary portion 350 leave spaces (such as spaces 355 and 355A) between the undulations of the continuous segments, thereby rendering the intermediary portion 350 as having an average width less than that of the widest section of each of the first and second end portions 312 and 322. More specifically, the second segment 345, the third segment 360 and the fourth segment 362 create an undulation that yields a space 355 in the width of plate 200 at that location. Note that because the undulations and resulting spaces of plate 300 extend almost half of the width of plate 300, as opposed to almost the entire width, each of space 355 and 355A is less than the spaces such as space 255 of plate 200. Consequently, because there is more material used in intermediary portion 350 than intermediary portion 250, if plate 300 is made of the same material as plate 200, the intermediary portion 350 of plate 300 would have less flexibility than the intermediary portion 250 of plate 200.

As stated, a variety of shapes of intermediary portions are contemplated and possible. As another example, an intermediary portion may be similar to that of FIG. 4C, but the corners where two continuous segments meet may be rounded instead of sharp. For example, FIG. 4D shows a partial, enlarged front view of another plate, which has an alternate intermediary portion 350D than that of plate 300. More specifically, instead of meeting at right angles to each other, the segments of intermediary portion 350D meet at angles that are less abrupt. That is, segments of intermediary portion 350D attempt to approximate a curved junction in that they have approximately 45 degree transition angles instead of the substantially 90 degree angles shown in intermediary portion 350 of plate 300.

Plate 300 demonstrates that the arrangement, shape or dimensions of any of the continuous segments of an intermediary portion may be varied to achieve desired physical characteristics of a plate. For example, the width throughout an intermediary portion of a plate need not be constant or based on a repeating pattern. The width and/or pattern may be varied to achieve desired results. For example, the segments on the right side of FIG. 4C may have a width different than that of the segments on the left side. Similarly, although the plates disclosed herein are relatively symmetric about the central longitudinal axis CLA, they may be asymmetric about the central longitudinal axis CLA.

As another example, the segments at one height or level along the central longitudinal axis CLA may differ from segments at a different height. Similarly, not only may the arrangement, shape or dimensions of any of the continuous segments of an intermediary portion be varied to achieve desired physical characteristics of a plate, but the first and second end portions may be altered or contain continuous segments similar to that of the disclosed intermediary portions. That is, a first end portion or second end portion may contain segments similar to those described herein for an intermediary portion. Further, an intermediary portion may have one design adjacent a first end portion, but a different design adjacent a second end portion. In this way, a plate may be asymmetric about its mid-point along the central longitudinal axis.

As yet another example, instead of two columns of segments (as shown in plate 300, for example, there is one column on the right side and one column on the left side), a plate may have more than two columns of segments. Further, the design of a plate may be adjusted or varied to meet certain mechanical limits. For example, a plate may be designed so as not to permit motion in a certain direction beyond a certain point. Further, a plate may be designed to have specific characteristics with respect to motion in a given direction such as flexion, extension, rotation or lateral bending, or any combination thereof. One example of this concept is illustrated with the intermediary portion 150I of FIG. 2I.

In addition, the material composition of a plate may affect physical characteristics. In some embodiments of a plate, the first end portion, the second end portion and the intermediary portion are made of a single material. In other embodiments of a plate, a combination of materials may be used to achieve desired results. For example, a plate may have an intermediary portion made of a different material than the first and second end portions. Further, as another example, an intermediary portion of a plate may itself be made of different materials. In addition, a plate with intermediary portions such as those disclosed herein may be covered or embedded in another material, for example, one that is softer than that of the intermediary portion. Such an embodiment may reduce the potential for any deleterious effects on any tissue adjacent the intermediary portion of such a plate.

In addition, note that plates such as plate 300 that have interior spaces within the side walls of the intermediary portion 350 or 350D. Such plates may further comprise material that covers and seals the intermediary portion so that such interior space may be closed or sealed so as to contain a compressible substance such as air or a compressible gas. In this way, for example, such a plate may perform in a fashion similar to a shock absorber.

One characteristic of some embodiments described herein is that one may use a relatively rigid material to construct a plate, but with the aid of the undulations of an intermediary portion, provide a degree of flexibility to the plate that otherwise may not be present. In the embodiments described herein, the various plates may be made of various biocompatible materials (metal or non-metal), including but not limited to, metals or a non-elastomeric polymers. Suitable materials may include, but not be limited to, Titanium alloys, commercially available Titanium, stainless steel, polyetheretherketone (“PEEK”), cobalt chrome (“CoCr”), polyetherketoneketone (“PEKK”), ultra high molecular weight polyethylene (“UHMWPE”), polyethylene, polyester (“PE), polyimide (“PI”), polyether imide (“PEI”), polyamide imide (“PAI”), polysulphone (“PSU”), polyether sulphone (“PES”), polyphenyl sulfone (“PPSU”), polyphenylene sulfide (“PPS”), polyphenylene oxide (“PPO”), liquid crystalline polyesters (“LCP”), syndiotactic polystyrene (“sPS”), polyacetal, polyamide (“PA”), polyether amide (“PEA”), cyclic olefin copolymer (“COC”), polypropylene (“PP”), polyethylene terephthalate (“PET”), polytetrafluoroethylene (“PTFE”), shape memory metals, other polymers or any combination of such materials. In addition, suitable materials may further include, but not be limited to, bioresorbable materials such as Poly-L-Lactide Acid (“PLLA”), Poly-L-Lactic-Co-Glycolic Acid (“PLGA”) and magnesium alloys such as AZ91. Further, any polymeric materials may include, for example, fillers of either a fibrous or particulate nature, plasticizers, chemical additives, or therapeutic agents to modify the physical properties, chemical stability, coloration, biostability or biocompatibility of a polymeric material used. With flexibility, the intermediary portions of the plates disclosed herein may act like springs. With some designs, the intermediary portions may behave in a linear manner. With some designs, the intermediary portions may behave in a non-linear manner.

As mentioned, the dimensions of a plate, or any portions thereof, may be modified to achieve desired physical characteristics. For illustration, a plate such as plate 100 may have the following dimensional characteristics: with an overall length of approximately 3.0 cm., the width 100W may be approximately 1.5 cm., the depth 100D may be 1.59 mm., the distance in the longitudinal direction (i.e., parallel to the central longitudinal axis CLA) of space 155 may be approximately 0.76 mm., the diameter of each fastener hole may be approximately 4.0 mm. and the distance in the longitudinal direction from the center of the first fastener hole 110 to the center of the second fastener hole 120 may be approximately 1.5 cm. A plate such as plate 200 may have the following dimensional characteristics: with an overall length of approximately 3.0 cm., the width 100W may be approximately 1.5 cm., the depth 100D may be 1.59 mm., the distance in the longitudinal direction of space 255 may be approximately 0.635 mm., the diameter of each fastener hole may be approximately 4.0 mm. and the distance in the longitudinal direction from the center of the first fastener hole 210 to the center of the second fastener hole 220 may be approximately 1.5 cm. A plate such as plate 300 may have the following dimensional characteristics: with an overall length of approximately 3.0 cm., the width 100W may be approximately 1.5 cm., the depth 100D may be 1.59 mm., the distance in the longitudinal direction of space 355 (or space 355A) may be approximately 0.635 mm., the diameter of each fastener hole may be approximately 4.0 mm. and the distance in the longitudinal direction from the center of the first fastener hole 310 to the center of the second fastener hole 220 may be approximately 1.5 cm. Further, note that plates 100, 200 and 300 are shown “to scale” in FIGS. 2, 2A, 2B, 3, 3A, 3B, 4, 4A and 4B, respectively, so that other approximate dimensions may be gleaned from the dimensions provided herein. In addition, note that a plate may have different depth (thickness) or other dimension based on its performance criteria. For example, a spinal plate for application in the cervical spine may have a depth of approximately 2.0 mm, whereas a spinal plate for application in another area of the spine may have a depth of approximately 5.0 mm.

Systems for connecting a first portion of bone and a second portion of bone comprise a plate such as those described herein and bone-engaging fasteners. For example, a system may comprise a plate with a single fastener hole at each of its end portions and first and second bone-engaging fasteners for engaging each fastener hole. Where such systems are used to connect vertebral bodies, the plate is a spinal plate and the fasteners may be, for example, bone screws. Further, a spinal plate such as those described herein may be utilized in a number of locations. For example, a spinal plate may be used in an anterior application such as an anterior cervical application. Also, it is contemplated that such plates may be utilized in anterior-lateral, lateral or posterior applications. For example, if used in a lateral application or posterior application, such plates may be in position suitable for addressing various lateral deformities. In addition, although the plates disclosed herein are described as connecting two portions of bone such as two adjacent vertebral bodies (i.e., single level plate), such plates also may be used to connect or bridge multiple levels of vertebral bodies (i.e., multi-level plates).

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. Furthermore, as used herein, the terms components and modules may be interchanged. It is understood that all spatial references, such as “superior,” “inferior,” “anterior,” “posterior,” “outer,” “inner,” and “perimeter” are for illustrative purposes only and can be varied within the scope of the disclosure.

What is claimed is:

1. A plate for connecting a first portion of bone and a second portion of bone, the plate comprising:
a central longitudinal axis;
a first end portion extending along the central longitudinal axis and configured to affix to the first portion of bone;
a second end portion extending along the central longitudinal axis and configured to affix to the second portion of bone;
an intermediary portion extending along the central longitudinal axis and between the first end portion and the second end portion, wherein the intermediary portion comprises three continuous segments of material, two segments of which extend along a direction substantially parallel to the central longitudinal axis, and one segment of which extends along a direction substantially perpendicular to the central longitudinal axis.

2. The plate of claim 1, wherein each of the first end portion, the second end portion and the intermediary portion have a width extending in a direction substantially perpendicular to the central longitudinal axis, and the one segment of the intermediary portion that extends along a direction substantially perpendicular to the central longitudinal axis extends along a direction substantially perpendicular to the width of the intermediary portion.

3. The plate of claim 1, wherein the plate comprises a first surface for engaging bone and a second surface opposing the first surface, and a depth defined by a distance between the first surface and the second surface, wherein the one segment of the intermediary portion that extends along a direction substantially perpendicular to the central longitudinal axis extends in a direction substantially parallel to the depth of the plate.

4. The plate of claim 1, wherein a cross section of the intermediary portion comprises a shape substantially similar to that of a square wave.

5. The plate of claim 1, wherein a cross section of the intermediary portion comprises a shape substantially similar to that of a sine wave.

6. The plate of claim 2, wherein the intermediary portion comprises a plurality of continuous segments of material, wherein:
a first segment extends along a direction substantially parallel to the central longitudinal axis, and a second segment extends along a direction substantially perpendicular to the central longitudinal axis and perpendicular to the width of the intermediary portion, wherein the second segment is connected to the first segment; and
a third segment extends along a direction substantially parallel to the central longitudinal axis, and a fourth segment extends along a direction substantially perpendicular to the central longitudinal axis and perpendicular to the width of the intermediary portion, wherein the third segment is connected to the second segment and wherein the fourth segment is connected to the third segment.

7. The plate of claim 6, wherein the intermediary portion further comprising fifth, sixth, seventh and eighth segments that are situated in the same manner as the first, second, third and fourth segments, respectively, wherein the fifth segment is connected to the fourth segment.

8. The plate of claim 3, wherein the intermediary portion comprises a plurality of continuous segments of material, wherein:
a first segment extends along a direction substantially parallel to the central longitudinal axis, and a second segment extends along a direction substantially perpendicular to the central longitudinal axis and substantially parallel to the depth of the plate, wherein the second segment is connected to the first segment; and
a third segment extends along a direction substantially parallel to the central longitudinal axis, and a fourth segment extends along a direction substantially perpendicular to the central longitudinal axis and substantially parallel to the depth of the plate, wherein the third segment is connected to the second segment and wherein the fourth segment is connected to the third segment.

9. The plate of claim 8, wherein the intermediary portion further comprises fifth, sixth, seventh and eighth segments that are situated in the same manner as the first, second, third and fourth segments, respectively, wherein the fifth segment is connected to the fourth segment.

10. The plate of claim 8, wherein:
the second segment extends away from the first segment in a direction away from the first surface of the plate; and
the fourth segment extends away from the third segment in a direction toward the first surface of the plate.

11. The plate of claim 10, wherein the first end portion has at least one fastener hole for receiving a first bone-engaging fastener, and wherein the second end portion has at least one fastener hole for receiving a second bone-engaging fastener.

12. A plate for connecting a first portion of bone and a second portion of bone, the plate comprising:
a first surface for engaging bone, a second surface opposing the first surface, and a depth defined by a distance between the first surface and the second surface;
a central longitudinal axis;
a first end portion extending along the central longitudinal axis and configured to affix to the first portion of bone;
a second end portion extending along the central longitudinal axis and configured to affix to the second portion of bone;
an intermediary portion extending along the central longitudinal axis and between the first end portion and the second end portion, wherein the intermediary portion comprises a plurality of continuous segments of material, wherein:
a first segment extends along a direction substantially parallel to the central longitudinal axis, and a second segment extends along a direction substantially perpendicular to the central longitudinal axis and substantially parallel to the depth of the plate, wherein the second segment is connected to the first segment; and
a third segment extends along a direction substantially parallel to the central longitudinal axis, and a fourth segment extends along a direction substantially perpendicular to the central longitudinal axis and substantially parallel to the depth of the plate, wherein the third segment is connected to the second segment and wherein the fourth segment is connected to the third segment.

13. The plate of claim 12, wherein the intermediary portion further comprises fifth, sixth, seventh and eighth segments that are situated in the same manner as the first, second, third and fourth segments, respectively, wherein the fifth segment is connected to the fourth segment.

14. The plate of claim 13, wherein:
the second segment extends away from the first segment in a direction away from the first surface of the plate;
the fourth segment extends away from the third segment in a direction toward the first surface of the plate;
the sixth segment extends away from the fifth segment in a direction away from the first surface of the plate; and
the eighth segment extends away from the seventh segment in a direction toward the first surface of the plate.

15. The plate of claim 12, wherein the first end portion, the second end portion and the intermediary portion are made of a single material.

16. The plate of claim 12, wherein at least the intermediary portion is made of non-elastomeric polymer.

17. The plate of claim 12, wherein at least the intermediary portion is made of metal.

18. The plate of claim 15, wherein the plate is made of non-elastomeric polymer.

19. A system for connecting a first portion of bone and a second portion of bone, the system comprising:
a plate comprising:
a first surface for engaging bone, a second surface opposing the first surface, and a depth defined by a distance between the first surface and the second surface;
a central longitudinal axis;
a first end portion extending along the central longitudinal axis and configured to affix to the first portion of bone, the first end portion having at least one fastener hole for receiving a first bone-engaging fastener;
a second end portion extending along the central longitudinal axis and configured to affix to the second portion of bone, the second end portion having at least one fastener hole for receiving a second bone-engaging fastener;
an intermediary portion extending along the central longitudinal axis and between the first end portion and the second end portion, wherein the intermediary portion comprises a plurality of continuous segments of material, wherein:
a first segment extends along a direction substantially parallel to the central longitudinal axis, and a second segment extends along a direction substantially perpendicular to the central longitudinal axis and substantially parallel to the depth of the plate, wherein the second segment is connected to the first segment; and
a third segment extends along a direction substantially parallel to the central longitudinal axis, and a fourth segment extends along a direction substantially perpendicular to the central longitudinal axis and substantially parallel to the depth of the plate, wherein the third segment is connected to the second segment and wherein the fourth segment is connected to the third segment.
a first bone-engaging fastener for engaging the at least one fastener hole in the first end portion and for engaging the first portion of bone;
a second bone-engaging fastener for engaging the at least one fastener hole in the second end portion and for engaging the second portion of bone.

20. The system of claim 19, wherein the system is used for connecting a first vertebral body and a second vertebral body, the plate is a spinal plate, and the first fastener is a bone screw and the second fastener is a bone screw.
